(19) United States
(12) Patent Application Publication
(10) Pub. No.: US 2024/0198135 A1
(43) Pub. Date: Jun. 20, 2024

(54) METHOD FOR GENERATING CONTROL DATA FOR AN IRRADIATION APPARATUS, COMPUTER PROGRAM PRODUCT, COMPUTER-READABLE STORAGE MEDIUM, ELECTRONIC COMPUTING FACILITY AND IRRADIATION APPARATUS

(71) Applicant: Siemens Healthcare GmbH, Erlangen (DE)

(72) Inventors: Hans Wehrl, Erlangen (DE); Matthias Fenchel, Erlangen (DE); Thomas Vahle, Nürnberg (DE)

(21) Appl. No.: 18/520,370

(22) Filed: Nov. 27, 2023

(30) Foreign Application Priority Data
Dec. 16, 2022 (EP) 22214198.8

(51) Int. Cl. A61N 5/10

(52) U.S. Cl. CPC A61N 5/1037; A61N 5/1039

(57) ABSTRACT

A method for generating control data for an irradiation apparatus for a patient by an electronic computing facility of the irradiation apparatus is provided. A movement model of the patient for irradiation in the irradiation apparatus is provided. Control data for the irradiation of the patient is generated in dependence on at least one item of patient information and in dependence on the movement model.

### RELATED APPLICATION

This application claims the benefit of EP 22214198.8, filed on Dec. 16, 2022, which is hereby incorporated by reference in its entirety.

### FIELD

The present disclosure relates to a method for generating control data for an irradiation apparatus for patients by an electronic computing facility of the irradiation apparatus. The present disclosure furthermore relates to a computer program product, a computer-readable storage medium, an electronic computing facility, and an irradiation apparatus.

### BACKGROUND

Radiotherapy is one of the most commonly used methods of cancer treatment. In many cases, external beam radiotherapy, also known as teletherapy or percutaneous radiotherapy, is used. The patient is generally placed in the vicinity of the linear accelerator (LINAC) and the target volume, for example the tumor, is fractionally irradiated for a few minutes, i.e., these treatment sessions are repeated over a period of days or weeks. During and between treatment sessions, the tissue in the patient's body can move relative to the reference point used for radiotherapy planning. Therefore, the planned target volume can move outside the radiation beam or healthy tissue can migrate into the radiation beam during treatment. Both effects have a negative impact on the outcome of the treatment because either the dose is too low for the target tissue or healthy tissue is irradiated and subsequently damaged, leading to side effects of the radiotherapy. Respiratory movements present one of the greatest challenges, in particular in the abdominal and thoracic regions, but other types of movement are also possible. Despite many approaches, movement during treatment remains a major challenge in radiotherapy, which also impairs the potential of techniques such as intensity-modulated radiotherapy (IMRT).

### SUMMARY

It is the object to provide a method, a computer program product, a computer-readable storage medium, an electronic computing facility, and a treatment apparatus by which improved irradiation of a patient is enabled.

One aspect relates to a method for generating control data for an irradiation apparatus for patients by an electronic computing facility of the irradiation apparatus. A movement model of the patient for treatment in the irradiation apparatus is provided, and control data for irradiation of the patient is generated in dependence on at least one item of patient information and in dependence on the movement model.

Hence, in particular, a method is presented for correspondingly predicting movement during irradiation as well and, in a further embodiment, also between the corresponding sessions. Herein, the problem arises not only in LINAC-based radiotherapy, but also with other types of radiotherapy, for example with a Gamma Knife. Advances in IMRT, which is even more accurate, focus even more on the challenges of patient movement during and between corresponding therapy sessions.

Hence, in particular, movement compensation can be performed on the basis of a movement model or also of a so-called digital twin of the patient during radiotherapy. Herein, in particular, a movement model is derived from a time-resolved scan, for example a magnetic resonance imaging scan, and a respiration model captured during the magnetic resonance imaging scan. This movement model is transferred to the radiotherapy treatment by capturing the respiratory signal during irradiation and adapting the movement model derived from the scan thereto. This supplies a movement model, for example of the internal organs and the target zone for irradiation that enables movement-corrected control of the irradiation system or the irradiation apparatus, for example the LINAC. Other sources of information, for example in the in-treatment X-ray image, can be additionally added in order to improve the accuracy of the model and the adaptation to the respective movement phase. Ideally, the patient is transported without changing position on a transfer bed, for example similar to a radiotherapy stretcher or bed, wherein, for example, a respiratory belt remains in situ as a signal transmitter.

Hence, in particular, the actual irradiation corresponds to radiotherapy. The patient information used can, for example, be the location of a tumor, corresponding tissue values, blood values, or the like. This enables treatment to be realized reliably, since control data generated on the basis of the method accordingly allows improved treatment.

Herein, the movement model is, in particular, based on an imaging method for the region to be treated. This imaging method can also be referred to as a scan.

According to an advantageous embodiment, the movement model is determined on the basis of a movement of the patient captured outside the irradiation apparatus at a time before the irradiation. Hence, in particular, the movement model is determined and provided at a time before the actual irradiation, for example in a magnetic resonance imaging system, so that essential real-time irradiation can be performed on the basis of the movement model.

Furthermore, it has been found to be advantageous for the movement model to be generated in the magnetic resonance imaging system on the basis of a movement of the patient determined by a magnetic resonance imaging system. A magnetic resonance imaging system is, in particular, suitable since a high-resolution imaging method/scan is used here. It is, for example, accordingly possible to record corresponding respiratory movements and the resulting movements within the body, for example of the organs, and to predict a corresponding movement of the same.

Moreover, it can be provided that a position of the patient in the magnetic resonance imaging system and a position of the patient in the irradiation apparatus are set at least substantially the same. It can, for example, be provided that the magnetic resonance imaging system is located in the vicinity of the actual irradiation apparatus, for example in the same room. For example, the magnetic resonance imaging system together with the irradiation apparatus can embody a corresponding irradiation system. It is then possible to use a corresponding bench/bed/stretcher in the magnetic resonance imaging system on which the patient is positioned and the corresponding movement model is generated. The patient can then be moved directly into the irradiation apparatus on the same bench and in the same position in order, for example, for the irradiation to be performed on the basis of the generated irradiation control data. This enables irradiation to be performed with high precision.

Furthermore, it has been found to be advantageous for respiration to be taken into account as the movement of the patient in the movement model. Respiration has corresponding impacts on the thoracic organs. Respiration cannot be stopped during irradiation so that movement of the organs is always recorded here. Respiration can now be used to create a prediction model for respiration and in particular for organ movement so that the control data can be correspondingly adapted to the respiration.

A further advantageous embodiment provides that a movement of organs of the patient is predicted by the movement model. Organ movement, in particular in the thoracic region, can, for example, now be predicted on the basis of the respiration so that reliable irradiation can be realized.

A further advantageous embodiment provides that an intensity of the irradiation, a position of the irradiation, and/or a time profile of the irradiation are set by the control data. This enables the performance of reliable irradiation since, for example, the intensity, in other words irradiation strength or position, can be adapted accordingly. In particular, this enables organ movement to be taken into account. In addition, it is also possible to set a time profile of the irradiation, for example the corresponding irradiation can be adapted in dependence on a respiration time profile so that only a so-called irradiation zone is irradiated.

Furthermore, it has been found to be advantageous for an irradiation zone for the irradiation to be specified and, at least for the irradiation zone, the movement model to be generated by the electronic computing facility. The irradiation zone is, in particular, a zone of the patient to which the radiotherapy, in other words the actual irradiation, is applied. It is now advantageous for, at least for the irradiation zone, the movement, for example of the organs, to be predicted so that advantageous irradiation of the irradiation zone can be performed.

A further advantageous embodiment provides that, during irradiation of the patient, current respiration of the patient is captured, and the control data is generated on the basis of the current respiration. For example, during irradiation, respiration can be captured by a respiratory belt and/or by a camera. Hence, it is possible for current respiration to be likewise taken into account and incorporated into the movement model, thus enabling the realization of an even more accurate prediction of movement, in particular of the organs. Hence, extremely efficient radiotherapy can be provided.

A further advantageous embodiment provides that, during irradiation, a respiratory signal for subsequent breathing is specified for the patient. For example, a respiration curve for subsequent breathing can be displayed visually so that the patient can subsequently breathe in accordance with the respiratory signal. Hence, this enables the prediction to be performed even more efficiently since the movement of the organs can be predicted even more precisely on the basis of the specified respiratory signal.

The method presented is, in particular, a computer-implemented method. Therefore, a further aspect relates to a computer program product with program code, which cause an electronic computing facility to perform a method according to the preceding aspect when the program code are processed by the electronic computing facility. The computer program product can also be referred to as a computer program.

Furthermore, the present disclosure also relates to a non-transitory computer-readable storage medium with at least the computer program product according to the preceding aspect.

A further aspect relates to an electronic computing facility (computer) for generating control data for an irradiation apparatus for a patient, wherein the electronic computing facility is embodied to perform a method according to the preceding aspect. In particular, the method is performed by the electronic computing facility.

The electronic computing facility has, for example, processors, circuits, in particular integrated circuits, and further electronic components in order to be able to carry out corresponding method acts.

Furthermore, the present disclosure also relates to an irradiation apparatus for irradiating a patient with at least one electronic computing facility according to the preceding aspect.

Moreover, the present disclosure also relates to an irradiation system with at least the irradiation apparatus and with a magnetic resonance imaging system. Preferably, the irradiation apparatus and the magnetic resonance imaging system can be embodied in the same room.

Independent of the grammatical term usage, individuals with male, female or other gender identities are included within the term.

For cases or situations which may arise with the use of the method and are not explicitly described here, it can be provided that, according to the method, an error message and/or a request for user feedback is output and/or a default setting and/or a predetermined initial state is set.

### BRIEF DESCRIPTION OF THE DRAWINGS

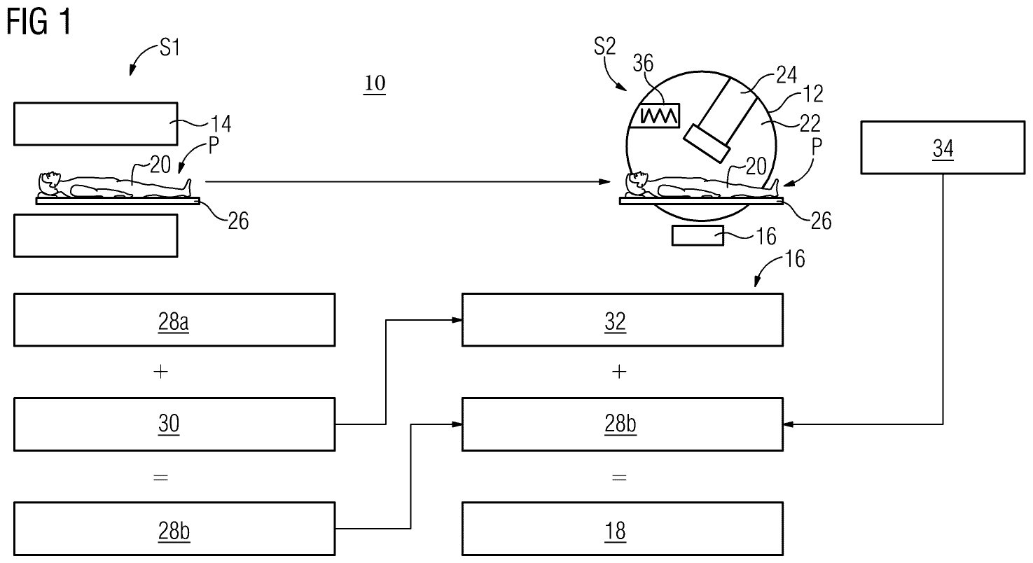

Further features and advantages can be found in the following description with reference to the attached FIG. 1. In FIG. 1, the same reference symbols denote the same features and functions. The exemplary embodiments only serve to explain the invention and are not intended to restrict the invention.

### DETAILED DESCRIPTION

The aspects are now described in more detail with reference to an exemplary embodiment in FIG. 1.

Herein, FIG. 1 shows a schematic block diagram according to one embodiment of the method.

FIG. 1 shows a schematic block diagram of an embodiment of an irradiation system 10 designated as a whole. The irradiation system 10 has at least one irradiation apparatus 12 and one magnetic resonance imaging system 14. The irradiation system 10 is preferably housed in a separate room.

The irradiation apparatus 12 has an electronic computing facility 16. The electronic computing facility 16 is embodied to generate control data 18 for the irradiation apparatus 12 for a patient 20. Herein, irradiation 22 is performed with an irradiation facility 24 of the irradiation apparatus 12.

In the exemplary embodiment shown in FIG. 1, the patient 20 is, for example, located on a stretcher 26, which, in a first act S1, is located in the magnetic resonance imaging system 14 and in a second act S2 is in turn located in the irradiation apparatus 12.

In a method presented for generating the control data 18, it is provided that a movement model 28a, 28b of the patient 20 is provided for treatment in the irradiation apparatus 12 and the generation of the control data 18 for irradiation of the patient is performed in dependence on at least one item of patient information and in dependence on the movement model 28a, 28b. In particular, FIG. 1 shows that a movement model 28a is generated for the entire patient 20, and a movement model 28b is in turn only provided for the organs of the patient 20. To generate the movement model 28b for the organs, in particular a respiratory signal 30 during the scan is also taken into account as an image recording of the patient 20 in the magnetic resonance imaging system 14.

In particular, it is furthermore shown that a current respiration 32 within the treatment apparatus 12 is likewise also captured and the control data 18 is in turn generated on the basis of the current respiration 32 and the movement model 28b of the organs. Furthermore, further information 34, for example X-ray images, can also be taken into account.

In particular, FIG. 1 shows that the movement model 28a, 28b is generated on the basis of the magnetic resonance imaging system 14 embodied outside the irradiation apparatus 12. Furthermore, FIG. 1 also shows that a position P of the patient 20 in the magnetic resonance imaging system 14 and a position P of the patient 20 in the irradiation apparatus 12 are at least set substantially the same.

In particular, it can furthermore be provided that the control data is used to set the intensity of the irradiation 22, the position of the irradiation 22, and/or a time profile of the irradiation 22.

Furthermore, it can be provided that the current respiration 32 is captured by a respiratory belt and/or a camera during irradiation 22 and/or respiration is captured in the magnetic resonance imaging system 14.

In addition, FIG. 1 shows that the irradiation apparatus 12 can also have a display facility 36, wherein the display facility 36 specifies a respiratory signal for the patient 20 during irradiation 22 for subsequent breathing. Hence, in particular, FIG. 1 shows a schematic representation of a possible embodiment of movement correction during irradiation 22, which can also be referred to as radiotherapy treatment, based on the imaging of the magnetic resonance imaging system 14 and a movement model 28a, 28b derived therefrom. A movement model 28a is derived from a time-resolved MRI scan and a respiratory signal captured during the MRI scan. This movement model 28a is in turn derived as a movement model 28b for the organs and transferred for the irradiation 22 in that the respiratory signal is captured during the treatment and the movement model 28a, 28b derived from the scan is adapted thereto. This, for example, provides a movement model 28b of the internal organs and the target zone for the irradiation 22 that enables motion-corrected control of the irradiation apparatus 12, for example the LINAC. Other sources of information, for example in the in-treatment X-ray image, can be added in order to improve the accuracy of the movement model 28a, 28b and the adaptation to the respective movement phase. Ideally, the patient 20 is transported without changing position on a transfer board, for example similar to an MRI table top, in the present case in particular the stretcher 26, wherein the respiratory belt remains in situ as a signal transmitter.

Furthermore, FIG. 1 in particular shows that the patient 20 is placed on the table top of the irradiation apparatus 12 and scanned in the magnetic resonance imaging system 14, which is located in the vicinity of the irradiation apparatus 12. This can be in the same hospital, department or even in the same room as the irradiation apparatus 12. The scan session serves to examine the region of interest, for example a tumor. The patient 20 is fitted with a respiratory belt in order to monitor and record a respiration curve. The output signal from this respiratory belt is fed into the magnetic resonance imaging system 14 and processed. The region of interest is also scanned at the same time. In an approach similar to the so-called Body Compass, this enables a sequence performed in free breathing to be ascertained. This magnetic resonance imaging sequence provides a movement-resolved four-dimensional image of the region of interest, wherein here, further magnetic resonance imaging sequences, for example with acceleration techniques or the like are possible. This makes it possible to correlate the individual movement states with the simultaneously captured respiratory signal. This information, for example positions of organs and regions of interest and the respiration curve formed with the respiratory belt, is then used as the basis for creating the movement model 28b of the organs.

When the scan has been completed, the patient 20 is moved to the irradiation apparatus 12, for example the LINAC. On the LINAC, the patient 20 is moved into the treatment position. Due to the stretcher 26, the position P in the treatment apparatus 12 can substantially coincide with the position P in the magnetic resonance imaging system 14. It may be necessary for the image of the magnetic resonance imaging system 14 to be co-registered with a type of, for example, portal X-ray image of the treatment apparatus 12 or with treatment apparatus planning images, ideally, this act can, for example, take place by an automatic algorithm. The respiratory signal is connected to the treatment apparatus 12. If the respiratory belt has not been removed from the patient 20 during the transfer and respiration to the monitoring unit has not been disconnected, it is, for example, also possible, to continue in the same respiratory phase, i.e., the respiratory signal is continuous from the scan to the actual treatment. Ideally, the patient 20 is transferred without the respiratory belt, cushions, and the like being moved so that the respiratory state can be monitored continuously. The movement model 28a, 28b derived from the scan images and the respiratory signal is continued in the treatment apparatus 12 and provides an accurate prediction of the organ and region of interest during irradiation 22. Therefore, the patient movement, in particular the respiratory movement, of the patient 20 during the treatment 22 can be predicted on the basis of the movement model 28b of the organs of the patient 20 using a relatively simple external respiratory signal.

Different sequences may be provided in the magnetic resonance imaging system 14, for example acceleration techniques, traces and the like, in order to obtain a better movement model 28a, 28b and information about the organs and the region of interest. In addition, it is possible to use various signal sources for the respiratory signal, for example, the respiratory band can be utilized by inductive or capacitive sensors, ultrasonic probes, ultra-wideband radar, or the like. The respiratory signal can be supplemented by other signals, such as, for example, sources for the heart rate or other movement sensors, for example a peristaltic movement derived from ultrasound. The respiratory signal can be decoupled from continuous monitoring during the sessions and the reconnection, in particular the phase of the respiration curve, is, for example, ascertained by an X-ray image recorded during or shortly before the irradiation 22. The respiratory signal during irradiation 22 can be supplemented by three-dimensional or four-dimensional formations obtained by X-ray imaging or other techniques during irradiation 22. The movement model 28a, 28b, which in particular corresponds to the digital twin of the patient 20, can be further enhanced by information from, for example, three-dimensional cameras or the like. Furthermore, adherence to a specific respiratory pattern, ideally the one used during the scan, is controlled by feedback to the patient, in particular visual feedback via a patient entertainment system/guidance system, for example the display facility 36.

Finally, the movement model 28b of the organs or the digital twin of the patient 20 can also be used not only to ensure movement compensation within a session for the treatment 22 but also to enable a comparison of movement and organs between individual treatment sessions on different days. For this purpose, either the scan can be repeated before each treatment session and the respective differences in the organ position between the movement models 28a, 28b derived from different days can be tracked and compensated, or the movement model 28a, 28b can be assumed to be constant and the comparison of subsequent sessions would, for example, take place via the respiratory signal obtained from the MRI device, in particular the treatment apparatus 12, and/or other guidelines in the treatment apparatus 12, such as the X-ray signal during the session. The in-session X-ray signal from the treatment apparatus 12 can also be used to compare the image data used for the movement model 28a, 28b and the coordinate frame if the transmission, for example on the stretcher 26, is not perfect.

Moreover, the movement model 28a, 28b or the digital twin can be used not only to turn the irradiation 22 on and off in order to compensate movement but can also be realized as an additional input parameter for modulating intensity, for example by calculating attenuation maps, synthetic CT maps from the movement model 28a, 28b.

The presented method in particular has the advantages that the movement model 28a, 28b is based on the individual patient and takes account of corresponding organ movements as opposed to external signals. It is possible to use a soft-tissue contrast of the MRI signal, which generally enables excellent identification of the target volume and the organs. Herein, there is no need for a simultaneous MRI LINAC system, which is technically complex, difficult for patient positioning, and expensive. The technique can be applied to a large number of already installed treatment apparatuses 12 and magnetic resonance imaging systems 14, i.e., it can be used on the basis of what is installed. Furthermore, it is possible to achieve excellent soft-tissue contrast and the ability to construct and derive movement models 28a, 28b, can be combined with existing treatment apparatus hardware or only requires a minimal change of hardware. Furthermore, it is possible to use a large number of different magnetic resonance imaging systems 14. Apart from the availability of the corresponding imaging sequence and of the trigger signal input, no special requirements for the magnetic resonance imaging system 14 are necessary. Herein, the magnetic resonance imaging system 14 does not have to be located in the same room or even in the same department as the irradiation apparatus 12, but this is preferably provided. In a first embodiment, a simple respiratory belt may suffice, thus minimizing expenditure on more complex detection mechanisms on the treatment apparatus 12. The applicability and transferal of an existing solution for PET/MR imaging minimizes development costs and enables rapid implementation and testing. In contrast to the stand-alone MRI solution for corresponding imaging with the magnetic resonance imaging system 14 which applies movement parameters for optimizing image quality later used for planning irradiation 22, this approach is concentrated on movement correction during treatment.

Although the invention has been illustrated and described in detail by way of the preferred exemplary embodiments, the invention is not restricted by the examples disclosed and other variations can be derived therefrom by a person skilled in the art without departing from the protective scope of the invention.

1. A method for generating control data for an irradiation apparatus for a patient, the method comprising:
providing a movement model of the patient for irradiation in the irradiation apparatus; and
generating control data for the irradiation of the patient in dependence on at least one item of patient information and in dependence on the movement model.

2. The method as claimed in claim 1, wherein the movement model is determined on the basis of a movement of the patient captured outside the irradiation apparatus at a time before the irradiation.

3. The method as claimed in claim 2, wherein the movement model is generated in a magnetic resonance imaging system on the basis of a movement of the patient determined by the magnetic resonance imaging system.

4. The method as claimed in claim 3, wherein a position of the patient in the magnetic resonance imaging system and a position of the patient in the irradiation apparatus are set the same.

5. The method as claimed in claim 1, wherein respiration is taken into account as a movement of the patient in the movement model.

6. The method as claimed in claim 1, wherein a movement of organs of the patient is predicted by the movement model.

7. The method as claimed in claim 1, wherein an intensity of the irradiation, a position of the irradiation, and/or a time profile of the irradiation are set by the control data.

8. The method as claimed in claim 1, wherein an irradiation zone for the irradiation is set and, at least for the irradiation zone, the movement model is generated by an electronic computing facility.

9. The method as claimed in claim 1, wherein, during the irradiation of the patient, a current respiration of the patient is captured, and the control data is generated on the basis of the current respiration.

10. The method as claimed in claim 9, wherein the current respiration is captured by a respiratory belt and/or by a camera during the irradiation.

11. The method as claimed in claim 1, wherein, during the irradiation, a respiratory signal for subsequent breathing is specified for the patient.

12. A non-transitory computer-readable storage medium with program code that, when executed by a computer, causes the computer to:
provide a movement model of the patient for irradiation in the irradiation apparatus; and
generate control data for the irradiation of the patient in dependence on at least one item of patient information and in dependence on the movement model.

13. The non-transitory computer-readable storage medium of claim 12, wherein the program code causes the computer to determine the movement model from a movement of the patient captured outside the irradiation apparatus at a time before the irradiation, wherein the movement model is generated in a magnetic resonance imaging system on the basis of a movement of the patient determined by the magnetic resonance imaging system, and wherein a position of the patient in the magnetic resonance imaging system and a position of the patient in the irradiation apparatus are set the same.

14. The non-transitory computer-readable storage medium of claim 12, wherein respiration is taken into account as a movement of the patient in the movement model, and wherein a movement of organs of the patient is predicted by the movement model, wherein, during the irradiation of the patient, a current respiration of the patient is captured, and the control data is generated on the basis of the current respiration.

15. The non-transitory computer-readable storage medium of claim 12, wherein an intensity of the irradiation, a position of the irradiation, and/or a time profile of the irradiation are set by the control data.

16. An irradiation apparatus for irradiating a patient, the irradiation apparatus comprising:
an irradiation source; and
a computer configured to generate control data for the irradiation of the patient in dependence on at least one item of patient information and in dependence on a movement model of the patient.

17. The irradiation apparatus of claim 16, wherein the movement model is generated in a magnetic resonance imaging system on the basis of a movement of the patient determined by the magnetic resonance imaging system.

18. The irradiation apparatus of claim 16, wherein the computer is configured to generate the control data accounting for respiration as a movement of the patient in the movement model, and wherein a movement of organs of the patient is predicted by the movement model, wherein, during the irradiation of the patient, a respiration sensor is configured to capture a current respiration of the patient, and the control data is generated on the basis of the current respiration.

19. The irradiation apparatus of claim 16, the computer is configured to generate the control data as an intensity of the irradiation, a position of the irradiation, and/or a time profile of the irradiation.
